(19) United States
(12) Patent Application Publication
(10) Pub. No.: US 2011/0166221 A1
(43) Pub. Date: Jul. 7, 2011

(54) USE OF DRONEDARONE FOR THE PREPARATION OF A MEDICAMENT FOR USE IN THE PREVENTION OF CARDIOVERSION

(75) Inventors: Christophe GAUDIN, Saint Cloud (FR); Nacera HAMDANI, Colombes (FR); Davide RADZIK, Paris (FR); Martin VAN EICKELS, Berlin (DE)

(73) Assignee: SANOFI-AVENTIS, Paris (FR)

(21) Appl. No.: 12/962,115

(22) Filed: Dec. 7, 2010

Related U.S. Application Data
(63) Continuation of application No. PCT/IB2009/006089, filed on Jun. 8, 2009.
(60) Provisional application No. 61/060,260, filed on Jun. 10, 2008.

(30) Foreign Application Priority Data
Jun. 10, 2008 (EP) 08290532.4

(51) Int. Cl. A61K 31/343; A61P 9/06

(52) U.S. Cl. 514/469

(57) ABSTRACT

Methods of using dronedarone in the prevention of cardioversion.

This application is a continuation of International Application No. PCT/IB2009/006089, filed Jun. 8, 2009, which is incorporated herein by reference in its entirety; which claims the benefit of U.S. Provisional Application No. 61/060,260, filed Jun. 10, 2008 and claims the benefit of priority of European Patent Application No. 08290532.4, filed Jun. 10, 2008.

The instant invention relates to the use of dronedarone for the preparation of a medicament for use in the prevention of cardioversion especially electrical cardioversion.

2-n-butyl-3-[4-(3-di-n-butylaminopropoxy)benzoyl]-5-methylsulfonamido-benzofuran or dronedarone and its pharmaceutically acceptable salts are described in European patent EP 0 471 609 B1.

Dronedarone is a multi-channel blocker that affects calcium, potassium and sodium channels and has anti-adrenergic properties.

Dronedarone is an anti-arrhythmic agent for the treatment of patients with a history of atrial fibrillation or atrial flutter.

Electrical therapy has been used to treat a variety of conditions since the 18th century and has led to the development of trans-thoracic direct current electrical cardioversion. It is now the treatment of choice for many cardiac arrhythmias.

Trans-thoracic electrical cardioversion has become the standard method for terminating AF since Lown first described it in 1962 (Lown B, Amarasingham R, Neuman J, et al: New method for treating cardiac arrhythmias; Use of synchronized capacitor discharge. JAMA 182: 548-555, 1962). Since then, this technique has been widely used and shown too effective. Research over the last decade has resulted in a better understanding of the mechanisms of defibrillation, the development of new technologies and energy waveforms, and novel optimization strategies to improve efficacy rates, patient safety, and success in refractory cases.

Electrical transthoracic cardioversion terminates arrhythmia by the delivery of a synchronized shock applied on the patient's chest via two paddles, it depolarizes the tissue involved in a reentrant circuit responsible for the arrhythmia. Depolarization of all involved excitable tissue of the circuit makes the tissue refractory, which is no longer able to propagate or sustain reentry.

According to the “Critical Mass Hypothesis” high defibrillation energy levels can eliminate fibrillatory activity. This theory hypothesizes that atrial or ventricular fibrillation is sustained by a certain amount of myocardium and terminated when the entire myocardium is uniformly depolarized (Zipes D P, Fischer J, King R M: Termination of ventricular fibrillation in dogs by depolarizing a critical amount of myocardium. Am J Cardiol 36:37-44, 1975).

The success of electrical cardioversion for the treatment of atrial fibrillation can be as high as 87%; it depends on the patient's conditions, in particular the duration of the atrial fibrillation episode as well as on intervention modalities. The position of the paddles used to apply the shock is particularly important; the anterior-lateral position with one paddle over the cardiac apex and one right infra-clavicular is the most effective (Botto G L, Politi A, Bonini W, et al: External cardioversion of atrial fibrillation: Role of paddle position on technical efficacy and energy requirements. Heart 82:726-730, 1999).

Although effective, the electrical cardioversion procedure can lead to sometimes severe complications, in particular thromboembolic events such as stroke and life-threatening cardiac arrhythmias.

Thromboembolic complications were reported in 1-7% of patients in particular in the absence of adequate anticoagulation (Bjerkelund C J, Orning O M: The efficacy of anticoagulant therapy in preventing embolism related to DC electrical conversion of atrial fibrillation. Am J Cardiol 23:208-216, 1969), (Arnold A Z, Mick M J, Mazurek R P, et al: Role of prophylactic anticoagulation for direct current cardioversion in patients with atrial fibrillation or atrial flutter. J Am Coll Cardiol 19:851-855, 1992).

Various arrhythmias may arise after cardioversion of atrial fibrillation, especially ventricular and supraventricular premature beats, bradycardia, and short periods of sinus arrest (Rabbino M D, et al: Complications and limitations of direct current countershock. JAMA 190:417-420, 1964). Electrolyte imbalances or treatment with digitalis may precipitate other dangerous arrhythmias, such as ventricular tachycardia and fibrillation (Lown B, Likoff W, Dreifus L S: Cardioversion and digitalis drugs: Changed threshold to electric shock in digitalized animals. Circ Res 17:519-531, 1965), (Aberg H, Cullhed I: Direct current countershock complications. Acta Med Scand 183:415-421, 1968).

Skin burns are also a frequent complication of electrical cardioversion (Pagan-Carlo, Stone M S, Kerber R E: Nature and determinants of skin burns after transthoracic cardioversion. Am J Cardiol 79:689-691, 1997).

Myocardial injury can also occur during electrical cardioversion (Lipkin D P, Frenneaux M, Stewart R, et al: Delayed improvement in exercise capacity after cardioversion of atrial fibrillation to sinus rhythm. Br Heart J 59:572-577, 1988), (Patton J N, Allen J D, Pantridge J F: The effects of shock energy, propranolol, and verapamil on cardiac damage caused by transthoracic countercheck. Circulation 69:357-368, 1984).

Furthermore electrical cardioversion is done under general anesthesia which by itself can lead to complications.

In conclusion, electrical cardioversion is commonly used for the treatment of atrial fibrillation. Although a highly successful therapy, it often needs to be repeated because of atrial fibrillation recurrences which can occur at any time. Each electrical cardioversion procedure carries a risk of complications which can be severe such as stroke or life-threatening cardiac arrhythmias.

For these reasons an agent which would decrease the need for electrical cardioversion would be of great benefit to atrial fibrillation patients.

The Inventors have now found that dronedarone decreases the need for cardioversion especially electrical cardioversion.

The subject of the instant invention is the use of dronedarone or one of its pharmaceutically acceptable salts for the preparation of a medicament for use in the prevention of cardioversion in patients with a history of atrial fibrillation or atrial flutter.

More precisely, the invention relates to the use of dronedarone or one of its pharmaceutically acceptable salts for the preparation of a medicament for use in the prevention of electrical cardioversion in patients with a history of atrial fibrillation or atrial flutter.

More precisely, the invention relates to the use of dronedarone or one of its pharmaceutically acceptable salts for the preparation of a medicament for use in the prevention of about 32% of cardioversion in patients with a history of atrial fibrillation or atrial flutter.

The percentage above corresponds to an average.

“Prevention of about 32%” means that a patient treated with dronedarone has a 32% lower risk of having a cardioversion compared to a patient not treated with dronedarone.

Among the pharmaceutically acceptable salts of dronedarone, mention may be made of the hydrochloride.

It will also be specified that the expression “having a history of atrial fibrillation or atrial flutter”, “with a history of or a current atrial fibrillation or flutter” or “with a recent history of or a current atrial fibrillation or flutter” or “with paroxysmal or persistent atrial fibrillation or flutter” or “with a history of, or a current paroxysmal or persistent atrial fibrillation or flutter” or “with a recent history of, or a current paroxysmal or persistent atrial fibrillation or flutter” means a patient who, in the past, has presented one or more episodes of atrial fibrillation or flutter and/or who is suffering from atrial fibrillation or atrial flutter at the time the dronedarone or a pharmaceutically acceptable salt thereof is used. More particularly, this expression means patients with documentation of having been in both atrial fibrillation or flutter and sinus rhythm within the last 6 months preceding the start of treatment. Patients could be either in sinus rhythm, or in atrial fibrillation or flutter at the time the dronedarone or a pharmaceutically acceptable salt thereof is initiated.

Among the patients having a history of atrial fibrillation or atrial flutter, mention may also be made of patients also exhibiting at least one of the following risk factors:
age notably equal to or above 70, or even above 75,
hypertension,
diabetes,
history of cerebral stroke or of systemic embolism,
left atrial diameter greater than or equal to 50 mm measured by echocardiography,
left ventricular ejection fraction less than 40%, measured by two-dimensional echography.
   Among patients with a history of atrial fibrillation or atrial flutter, mention may also be made of patients having additional risk factors corresponding to at least one of the following diseases:
hypertension,
structural heart disease,
tachycardia,
coronary heart disease,
non-rheumatic valvular heart disease,
ischemic dilated cardiomyopathy,
a history of ablation for AF/AFL for example catheter ablation or surgical ablation,
supra-ventricular tachycardia other than AF/AFL,
history of cardiac valve surgery,
non-ischemic dilated cardiomyopathy,
hypertrophic cardiomyopathy,
rheumatic valvular heart disease,
sustained ventricular tachycardia,
congenital heart disease,
a history of ablation for other reason than AF/AFL for example catheter ablation,
ventricular fibrillation,
   and/or at least a cardiac device chosen among:
a pacemaker,
an implanted cardioverter defibrillator.

Another object of the invention is a pharmaceutical composition which comprises, as active principle, dronedarone or a pharmaceutically acceptable salt thereof according to the present invention. This pharmaceutical composition comprises an effective dose of at least dronedarone, or an addition salt thereof with a pharmaceutically acceptable salt, or a hydrate or solvate thereof, and at least one pharmaceutically acceptable excipient. Said excipients are chosen according to the pharmaceutical form and the administration route desired, among usual excipients known to one of skill in the art.

In the pharmaceutical compositions according to the invention for the oral, sublingual, sub-cutaneous, intramuscular, intra-venous, topical, local, intratracheal, intranasal, transdermal or rectal administration, dronedarone or its salt, solvate or hydrate, can be administered as a unitary dosage form, in blend with usual pharmaceutical excipients, to animals and human beings for the prevention or for the treatment of pathological states mentioned above. The appropriate unitary dosage forms comprise the oral forms, such as tablets, hard or soft gelatin capsules, powders, granules and oral solutions or suspensions, the sublingual, buccal, intratracheal, intraocular, intranasal forms, the forms adapted to inhalation, topical, transdermal, sub-cutaneous, intramuscular or intra-venous delivery, the rectal forms and the implants. For the topical application, the compounds of the invention may be used as creams, gels, ointments or lotions.

For their therapeutic use, dronedarone and pharmaceutically acceptable salts thereof are generally introduced into pharmaceutical compositions.

These pharmaceutical compositions contain an effective dose of dronedarone or of a pharmaceutically acceptable salt thereof, and also at least one pharmaceutically acceptable excipient.

Said excipients are chosen according to the pharmaceutical form and the administration route desired, among usual excipients known of one of skill in the art.

Said pharmaceutical composition may be given once or twice a day with food.

The dose of dronedarone administered per day, orally, may reach 800 mg, taken in one or more intakes, for example one or two.

More specifically, the dose of dronedarone administered may be taken with food.

More specifically, the dose of dronedarone administered per day, orally, may reach 800 mg, taken in two intakes with a meal.

The dose of dronedarone administered per day, orally may be taken at a rate of twice a day with a meal for example with the morning and the evening meal.

More specifically, the two intakes may comprise same quantity of dronedarone.

In the pharmaceutical compositions for the oral, sublingual, sub-cutaneous, intramuscular, intra-venous, topical, local, intratracheal, intranasal, transdermal or rectal administration, dronedarone or one of its pharmaceutically acceptable salts, can be administered as a unitary dosage form, in blend with usual pharmaceutical excipients, to animals and human in diseases above mentioned.

The appropriate unitary dosage forms comprise the oral forms, such as tablets, hard or soft gelatin capsules, powders, granules and oral solutions or suspensions, the sublingual, buccal, intratracheal, intraocular, intranasal forms, by inhalation, the topical, transdermal, sub-cutaneous, intramuscular or intra-venous forms, the rectal forms and the implants. For the topical application, the compounds of the invention may be used as creams, gels, ointments or lotions.

As an example, a unitary dosage form for dronedarone or one of its pharmaceutically acceptable salts, in the form of a tablet, can comprise the following ingredients:

| Ingredients | mg |
|---|---|
| dronedarone hydrochloride (corresponding to | 426 |
| 400 mg of base) | |
| Methylhydroxypropylcellulose | 21.1 |
| Lactose monohydrate | 46.55 |
| Modified corn starch | 45.5 |
| Polyvinylpyrrolidone | 65 |
| Poloxamer 407 | 40 |
| Anhydrous colloidal silica | 2.6 |
| magnesium stearate | 3.25 |
| | 650 |
| dronedarone hydrochloride (corresponding to | 426 |
| 400 mg of base) | |
| microcrystalline cellulose | 65 |
| Anhydrous colloidal silica | 2.6 |
| anhydrous lactose | 42.65 |
| Polyvinylpyrrolidone | 13 |
| Poloxamer 407 | 40 |
| Macrogol 6000 | 57.5 |
| magnesium stearate | 3.25 |
| | 650 |
| dronedarone hydrochloride (corresponding to | 426 |
| 400 mg of base) | |
| microcrystalline cellulose | 26 |
| corn starch | 45.5 |
| Polyvinylpyrrolidone | 65 |
| Poloxamer 407 | 40 |
| Anhydrous colloidal silica | 3.25 |
| magnesium stearate | 3.25 |
| Lactose monohydrate | 41.65 |
| | 650 |
| dronedarone hydrochloride (corresponding to | 213 |
| 400 mg of base) | |
| microcrystalline cellulose | 13 |
| corn starch | 22.75 |
| Polyvinylpyrrolidone | 32.5 |
| Poloxamer 407 | 20 |
| Anhydrous colloidal silica | 1.3 |
| magnesium stearate | 1.625 |
| Lactose monohydrate | 20.825 |
| | 650 |

For oral administration, dronedarone daily dose may reach 800 mg.

In specific cases, higher or lower dosages may be appropriated; these dosages are comprised within the scope of the present invention. According to usual practice, the dosage suitable to each patient is determined by the physician according to the administration route, the weight, the disease, the body surface, the cardiac output and response of the patient.

The instant invention also relates to a method of treatment of the above mentioned disease which comprises the administration to a patient of an effective dose of at least dronedarone or one of its pharmaceutically acceptable salts.

### BRIEF DESCRIPTION OF THE DRAWINGS

The invention is illustrated with the above data with reference to the following FIGURE:

FIG. 1 represents Kaplan Meier cumulative incidence curves from randomization to first cardioversion during the on-study period of lasting from 12-30 months for the individual patients.

Efficacy of dronedarone and its pharmaceutically acceptable salts versus placebo for the prevention of cardioversion was provided via dronedarone hydrochloride during a prospective, multinational, double-blind, randomized, multi-center, placebo-controlled, parallel group trial.

### I. Selection of Patients

Patients must have a history of atrial fibrillation or atrial flutter and/or may be in normal sinus rhythm or in atrial fibrillation or flutter at the time of recruitment.

Recruitment of patients was conducted taking into account the following inclusion criteria:

### Inclusion Criteria:

1) One of the following risk factors had to be present:
   age equal to or greater than 70 years,
   hypertension (taking antihypertensives of at least two different classes),
   diabetes,
   history of cerebral stroke (transient ischemic event or completed cerebral stroke) or of systemic embolism,
   left atrial diameter greater than or equal to 50 mm measured by echocardiography,
   left ventricular ejection fraction less than 40%, measured by two-dimensional echography;
or
   age equal to or above 70, or even above 75, possibly combined with at least one of the risk factors below:
      hypertension (taking antihypertensives of at least two different classes),
      diabetes,
      history of cerebral stroke (transient ischemic event or completed cerebral stroke) or of systemic embolism,
      left atrial diameter greater than or equal to 50 mm measured by echocardiography,
      left ventricular ejection fraction less than 40%, measured by two-dimensional echography;
2) availability of one electrocardiogram within the last six months, showing that the patients was or is in atrial fibrillation/flutter,
3) availability of one electrocardiogram within the last six months, showing that the patients was or is in sinus rhythm.

### II. Duration and Treatment

Study drug treatment units (placebo or dronedarone hydrochloride corresponding to 400 mg of base) were such that each patient took one tablet in the morning during or shortly after breakfast and one tablet in the evening during or shortly after dinner.

The treatment duration depended on the time of recruitment of each patient in the trial and could be comprised from 12 months to 30 months.

### III. Results

Results were calculated using non-parametric Kaplan-Meier estimate.

Cox's proportional hazard model was used to estimate the hazard ratio also called relative risk.

Relative risk (RR) is the ratio between the risk of having a cardioversion for patients treated with dronedarone and the risk of having a cardioversion for patients treated with placebo.

Percentage of decrease of an event is calculated as follow:

x=1−RR.

### Results Relating to Cardioversion

From the 4628 patients included in the trial, 2301 were part of the group treated with dronedarone hydrochloride.

481 events were registered in the placebo group versus 339 in the group treated with dronedarone hydrochloride.

Calculated relative risk was equal to 0.68, i.e. a decrease of electrical cardioversion of 32% (p<0.001).

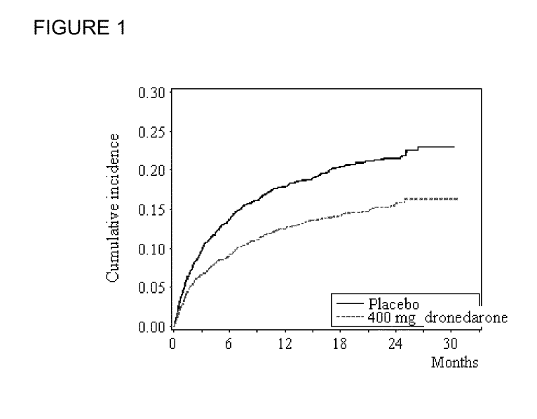

FIG. 1 shows that the effect of dronedarone occurred early and increased over time.

What is claimed is:

1. A method of preventing cardioversion in a patient comprising administering to the patient an effective dose of dronedarone or a pharmaceutically acceptable salt thereof.

2. The method according to claim 1, wherein the cardioversion is electrical cardioversion.

3. The method according to claim 1, wherein about 32% of cardioversion is prevented.

4. The method according to claim 1, wherein the patient has a history of atrial fibrillation or atrial flutter.

5. The method according to claim 2, wherein the patient has a history of atrial fibrillation or atrial flutter.

6. The method according to claim 3, wherein the patient has a history of atrial fibrillation or atrial flutter.

7. The method according to claim 1, wherein the patient has at least one risk factor selected from the group consisting of:
age notably equal to or above 70, or even above 75,
hypertension,
diabetes,
prior cerebrovascular accident or systemic embolism,
left atrium diameter greater that or equal to 50 mm by echocardiography, and
left ventricular ejection fraction less than 40% by 2D-echocardiography.

8. The method according to claim 2, wherein the patient has at least one risk factor selected from the group consisting of:
age notably equal to or above 70, or even above 75,
hypertension,
diabetes,
prior cerebrovascular accident or systemic embolism,
left atrium diameter greater that or equal to 50 mm by echocardiography, and
left ventricular ejection fraction less than 40% by 2D-echocardiography.

9. The method according to claim 3, wherein the patient has at least one risk factor selected from the group consisting of:
age notably equal to or above 70, or even above 75,
hypertension,
diabetes,
prior cerebrovascular accident or systemic embolism,
left atrium diameter greater that or equal to 50 mm by echocardiography, and
left ventricular ejection fraction less than 40% by 2D-echocardiography.

10. The method according to claim 4, wherein the patient has at least one risk factor selected from the group consisting of:
age notably equal to or above 70, or even above 75,
hypertension,
diabetes,
prior cerebrovascular accident or systemic embolism,
left atrium diameter greater that or equal to 50 mm by echocardiography, and
left ventricular ejection fraction less than 40% by 2D-echocardiography.

11. The method according to claim 5, wherein the patient has at least one risk factor selected from the group consisting of:
age notably equal to or above 70, or even above 75,
hypertension,
diabetes,
prior cerebrovascular accident or systemic embolism,
left atrium diameter greater that or equal to 50 mm by echocardiography, and
left ventricular ejection fraction less than 40% by 2D-echocardiography.

12. The method according to claim 6, wherein the patient has at least one risk factor selected from the group consisting of:
age notably equal to or above 70, or even above 75,
hypertension,
diabetes,
prior cerebrovascular accident or systemic embolism,
left atrium diameter greater that or equal to 50 mm by echocardiography, and
left ventricular ejection fraction less than 40% by 2D-echocardiography.

13. The method according to any one of claims 1 to 12, wherein the patient has an additional risk factor corresponding to at least one disease selected from the group consisting of:
hypertension,
structural heart disease,
tachycardia,
coronary heart disease,
non-rheumatic valvular heart disease,
ischemic dilated cardiomyopathy,
ablation for AF/AFL,
supra-ventricular tachycardia other than AF/AFL,
history of cardiac valve surgery,
non-ischemic dilated cardiomyopathy,
hypertrophic cardiomyopathy,
rheumatic valvular heart disease,
sustained ventricular tachycardia,
congenital heart disease,
ablation for other reason than AF/AFL, and
ventricular fibrillation,
and/or at least a cardiac device chosen among:
a pacemaker, and
an implanted cardioverter defibrillator.

14. The method according to any one of claims 1 to 12, wherein the dronedarone or pharmaceutically acceptable salt thereof is administered orally and wherein the dronedarone daily dose is up to 800 mg.

15. The method according to claim 13, wherein the dronedarone or pharmaceutically acceptable salt thereof is administered orally and wherein the dronedarone daily dose is up to 800 mg.
